(19) United States
(12) Patent Application Publication
(10) Pub. No.: US 2009/0209737 A1
(43) Pub. Date: Aug. 20, 2009

(54) USE OF DENATURING AGENTS DURING AFFINITY CAPTURE

(75) Inventor: Laura Bradbury, Newton, MA (US)

Correspondence Address: LEYDIG VOIT & MAYER, LTD, 700 THIRTEENTH ST. NW, SUITE 300, WASHINGTON, DC, 20005-3960, US

(73) Assignee: PALL CORPORATION, East Hills, NY (US)

(21) Appl. No.: 12/307,596

(22) PCT Filed: Jul. 5, 2007
(86) PCT No.: PCT/US07/72837

§ 371 Date: Jan. 6, 2009

Related U.S. Application Data
(60) Provisional application No. 60/818,957, filed on Jul. 7, 2006.

(51) Int. Cl. C07K 1/22; B01L 3/00

(52) U.S. Cl. 530/413; 422/61

(57) ABSTRACT

A method for affinity capturing target molecules is disclosed, comprising obtaining a sample comprising a target molecule; and, in the presence of a denaturing agent; affinity capturing the target molecule with a single chain affinity molecule or antigen binding portion thereof that specifically binds the target molecule, wherein the affinity molecule or antigen binding portion thereof is immobilized on a support.

### CROSS-REFERENCE TO RELATED APPLICATIONS

This patent application claims the benefit of U.S. Provisional Patent Application No. 60/818,957, filed Jul. 7, 2006, which is incorporated by reference.

### BACKGROUND OF THE INVENTION

Affinity capture protocols, e.g., capturing target proteins with antibody or antibody-like molecules coupled or bound to a solid support, are known in the art. These protocols can include a single recognition capture (e.g., immunoprecipitation) or more than one recognition capture (e.g., a sandwich assay). Since non-specific binding (e.g., of proteins including the target protein in a sample to materials such as beads or plastics that the protein contacts, or interaction between the non-target protein and the target protein) can adversely impact the results, these protocols attempt to reduce non-specific binding by, for example, capturing the protein with the antibody or antibody-like molecule (bound to the support), and subsequently washing the support (with the captured protein) with gentle detergents or salts in the wash buffer to remove the non-specifically bound material. The detergents or salts will not denature or significantly alter the target protein structure, which could lead to protein loss. However, extensive washing generally leads to some loss of target protein.

The present invention provides for ameliorating at least some of the disadvantages of the prior art. These and other advantages of the present invention will be apparent from the description as set forth below.

### BRIEF SUMMARY OF THE INVENTION

An embodiment of the invention provides a method for affinity capture of a target molecule comprising affinity capturing the target material(s) with a single-chain affinity molecule or an antigen binding portion thereof immobilized on a support, in the presence of at least one denaturing agent. A preferred embodiment of the invention comprising affinity capturing a target protein with an immobilized camelid antibody or antigen binding portion thereof, in the presence of at least one denaturing agent.

Another embodiment of the invention provides a kit for affinity capture of a target molecule comprising a support; a single-chain affinity molecule or antigen portion thereof, immobilized on the support; at least one buffer; and, a device suitable for containing the immobilized affinity molecule or antigen binding portion thereof. Typically, the device comprises a spin device, a multiple well plate (the multiple well plate can comprise a spin device) and/or a chromatography column. Preferably, the kit includes an insert comprising written instructions for using the kit.

In one embodiment, the kit includes a biochip comprising the single-chain affinity molecule immobilized on the support

### DETAILED DESCRIPTION OF THE INVENTION

In accordance with an embodiment of the present invention, a method for affinity capture of a target molecule comprises obtaining a sample comprising a target molecule; and, in the presence of a denaturing agent; affinity capturing the target molecule with a single-chain affinity molecule or antigen binding portion thereof that specifically binds the target molecule, wherein the affinity molecule is immobilized on a support.

In another embodiment of the invention, a kit for affinity capture of a target molecule comprises a support; a single-chain affinity molecule or antigen binding portion thereof, immobilized on the support; at least one buffer; and, a device suitable for containing the immobilized affinity molecule or antigen biding portion thereof.

In a preferred embodiment, the single-chain affinity molecule is a camelid antibody or an antigen binding portion thereof.

Without being limited to any particular mechanism, it is believed the denaturing agent changes the conformation (e.g., the 3D structure) of the target molecule(s) and other molecules in the sample, and thus prevents or minimizes binding between the target molecule(s) and the non-target molecules, and prevents or minimizes binding of the non-target molecules to the support and the affinity molecule. However, while it is believed the denaturing agent changes the conformation of the target molecule, the affinity molecule maintains the ability to specifically bind the target molecule present in the sample.

Also, again without being limited to any particular mechanism, it is believed nonspecific binding is prevented, rather than disrupted after it has occurred.

Advantageously, embodiments of the invention provide a method, kit, and system for reducing non-specific binding that does not require extensive washing to remove non-specifically bound materials, e.g., non-specifically bound proteins.

Each of the components of the invention will now be described in more detail below.

In contrast with a naturally occurring antibody (e.g., IgG) that includes four polypeptide chains, two heavy (H) chains and two light (L) chains inter-connected by disulfide bonds, the affinity molecule according to preferred embodiments of the invention comprises all or part of a single-chain antibody, more preferably, a heavy chain antibody, even more preferably, a camelid antibody.

In another embodiment, the affinity molecule can comprise a single protein chain, known as single chain Fv (scFv) (Huston et al., Proc. Natl. Acad. Sci. USA, 85, 5879-5883 (1988)).

Camelid antibodies (e.g., from camels, dromedaries, or llamas) are devoid of light chains, and the heavy chains bind their antigen by one single domain, the variable domain of the heavy immunoglobulin chain, referred to as VHH. VHHs show homology with the variable domain of heavy chains of the VHIII family (Dumoulin et al., Protein Science, 11:500-515 (2002)).

Suitable camelid antibodies (e.g., having desired binding specificities) and fragments (i.e., having an antigen binding portion) thereof can be produced, and immobilized on supports, as is known in the art (e.g., ten Haaft et al., “Separation in Proteomics: Use of Camelid Antibody Fragments in the Depletion and Enrichment of Human Plasma Proteins for Proteomics Applications,” in Separation Methods in Proteomics, Smejkal and Lazarev (ed.) CRC Press, 29-40 (2005)). Suitable camelid antibodies are also commercially available, e.g., from BAC B.V. (Naarden, the Netherlands) under the tradename CAPTURESELECT®.

The antibody or fragment thereof specifically binds or specifically immunoreacts with the target molecule antigen. By “specifically binds” is meant that the antibody binding is non-random, and the antibody differentially (or preferentially) binds the target molecule compared to an unrelated biological moiety. The antibody or fragment thereof can have any level of affinity or avidity for the antigen.

The affinity molecule can be immobilized on a variety of supports. Suitable supports include, for example, beads or irregular particles (e.g., in size of about 0.1 mm diameter or larger, typically ranging in size from about 5 microns to about 500 microns in diameter). The beads or particles can form a chromatography medium that one can use to pack a chromatography column. Alternatively, the support can be in the form of fibers (hollow or otherwise), membranes, or sponge-like materials permeated with holes in, for example, the micron to multi-millimeter sizes.

In yet another embodiment, the support comprises a solid substrate, providing a “biochip” or microarray format, where the substrate presents a generally planar surface to which is attached the affinity molecule or antigen binding portion thereof. In certain embodiments, the solid support can be transparent. The biochip can be a mass spectrometer probe. Preferred solid supports in this context include a metal, metal oxide, silicon, glass, a polymer (e.g., an organic polymer such as plastic), and a composite material. The surfaces of these supports can be modified for linking the affinity molecule or binding portion as is known in the art. Suitable metals include, for example, gold, aluminum, iron, titanium, chromium, platinum, copper and their respective alloys. Such metals can be derivatized on their surfaces with silicon dioxide, for instance, to provide reactive groups for linking. One method of derivatizing a metal surface is to sputter a metal oxide, such as silicon oxide, onto the metal surface.

In accordance with embodiments of the invention, the support may comprise an organic material. Exemplary organic materials are polysaccharides, such as cellulose, starch, agar, agarose, and dextran. Hydrophilic synthetic polymers are contemplated, including substituted or unsubstituted polyacrylamides, polymethacrylamides, polyacrylates, polymethacrylates, polyvinyl hydrophilic polymers, polystyrene, polysulfone, and copolymers or styrene and divinylbenzene. Alternatively, inorganic materials may be used as the solid support material. Such inorganic materials include but are not limited to porous mineral materials, such as silica; hydrogel-containing silica, zirconia, titania, alumina; and other ceramic materials. It is also possible to use mixtures of these materials, or composite materials, e.g., formed by copolymerization of or by an interpenetrated network of two materials, such as those disclosed in U.S. Pat. No. 5,268,097, No. 5,234,991, and No. 5,075,371.

Other suitable polymers for use as supports, particularly supports comprising membranes, include polyaromatics, polysulfones (including aromatic polysulfones such as polyethersulfone, bisphenol A polysulfone, and polyphenylsulfone), polyamides, polyimides, polyolefins, polystyrenes, polycarbonates, cellulosic polymers such as cellulose acetates and cellulose nitrates, fluoropolymers, and PEEK.

A variety of denaturing agents can be used individually, sequentially, or in combination, in accordance with embodiments of the invention.

The denaturing agent may comprise, for example, one or more chaotropic agent(s), lyotropic agent(s), organic denaturant(s), and/or detergent(s). Preferably, in those embodiments wherein the denaturing agent includes a detergent, the denaturing agent also includes one or more chaotropic agent(s), lyotropic agent(s), and/or organic denaturant(s), e.g., the denaturing agent further comprises a detergent, in addition to a chaotropic agent, lyotropic agent and/or organic denaturant.

Chaotropic agents may include a variety of different compounds, such as, for example, urea, CNS⁻, and CCl₃COO⁻, guanidine HCl, NO₃⁻, and ClO₄⁻.

Lyotropic agents may include, for example, SO₄²⁻, HPO₄²⁻, and acetate (CH₃COO⁻), e.g., sodium acetate (NaOAc).

Organic denaturants may include, for example, acetonitrile (ACN).

Detergents may include anionic, cationic, nonionic, or zwitterionic, detergent(s).

Anionic detergents may include, for example, deoxycholic acid, cholic acid, and, less desirably, SDS (sodium dodecyl sulfate); cationic detergents may include, for example, cetyltrimethylammonium bromide (CTAB). Nonionic detergents may include, for example, digitonin, triton, tween and nonidet 40 (NP40); Zwitterionic detergents may include, for example, CHAPS, CHAPSO, BigCHAP, CHAPS, ZWITTERGENT 3-08, ZWITTERGENT 3-10, ZWITTERGENT 3-12, ZWITTERGENT 3-14, and ZWITTERGENT 3-16.

Preferably, the denaturing agent is utilized with a buffer, e.g., to provide a denaturant fluid comprising at least one denaturing agent. A variety of buffers are suitable, for example, zwitterionic, phosphate, acetate, and carbonate. Zwitterionic buffers may include, for example, Tris buffer. Phosphate buffers, e.g., phosphate buffer solutions, may include, for example, sodium phosphate and potassium phosphate buffers.

In an embodiment, the denaturing agent is selected from the group consisting of urea, CHAPS, guanidine HCl, CTAB, acetate, and acetonitrile.

In some embodiments wherein at least one denaturing agent is urea, the urea has a concentration of at least about 0.8M, or at least about 1M, when placed in contact with the sample and/or the affinity molecule. For example, the urea may have a concentration in the range of from about 1M to about 9 M, or in the range of from about 1M to about 6M, when placed in contact with the sample and/or the affinity molecule.

In some embodiments wherein at least one denaturing agent is CHAPS, the CHAPS has a concentration of at least about 0.1%, or at least about 0.25%, when placed in contact with the sample and/or the affinity molecule. For example, the CHAPS may have a concentration in the range of from about 0.25% to about 2% when placed in contact with the sample and/or the affinity molecule.

In some embodiments wherein at least one denaturing agent is guanidine HCL, the guanidine HCl has a concentration of at least about 0.03M, or at least about 0.05M, when placed in contact with the sample and/or the affinity molecule. For example, the guanidine HCl can have a concentration in the range of from about 0.05M to about 2M when placed in contact with the sample and/or the affinity molecule.

In some embodiments wherein at least one denaturing agent is acetonitrile, the acetonitrile has a concentration of at least about 8%, or at least about 10%, when placed in contact with the sample and/or the affinity molecule. For example, the acetonitrile can have a concentration in the range of from about 10% to about 40% when placed in contact with the sample and/or the affinity molecule.

In some embodiments wherein at least one denaturing agent is acetate, the acetate has a concentration of at least about 30 mM, or at least about 50 mM, when placed in contact with the sample and/or the affinity molecule. For example, the acetate can have a concentration in the range of from about 50 mM to about 200 mM when placed in contact with the sample and/or the affinity molecule.

As noted above, denaturing agents can be used individually, sequentially, or in combination, e.g., two or more agents sequentially, or in combination, in some embodiments, three or more agents sequentially, or in combination. For example, in one embodiment utilizing at least two agents, a chaotropic agent (e.g., urea) is utilized in combination with a detergent (e.g., CHAPS), in a buffer.

The concentration of denaturing agent(s) placed in contact with the target molecule optionally may be adjusted to optimize the denaturation of the target molecule and/or the reduction of non-specific binding.

For example, an initial high concentration of denaturing agent can be combined with a target molecule containing-sample, and the concentration of the denaturing agent may be reduced in subsequent dilutions. As an illustrative example, a sample of human serum may be combined with a denaturing agent containing 9 M urea and 2% CHAPS to obtain a urea concentration of 5 M and a CHAPS concentration of 1.1%. The treated serum may then be diluted with phosphate buffered saline (PBS) to obtain a denaturing agent concentration of 2.25 M urea and 0.5% CHAPS. An initially high concentration of denaturing agent may advantageously denature denaturation-resistant target molecules present in the sample.

The application of the sample containing target molecules to the affinity capture media may possibly also reduce the concentration of the denaturing agent. For example, the application of the sample to affinity capture media, which may comprise, e.g., buffer solution, may reduce the concentration of the denaturing agent. However, such a reduction would not significantly compromise the denaturation of the target molecule(s). As an illustrative example, a slurry of affinity capture beads and buffer solution provides a fluid that may be centrifuged in a spin device (e.g., a NANOSEP® device), leaving little or no fluid between the beads and/or within the beads. Any fluid remaining in and/or between the beads may slightly reduce the concentration of denaturing agent, but may not significantly compromise the denaturation of the target molecule(s) in the sample.

The affinity capture media may also, optionally, be equilibrated prior to the application of the sample to the media so that the affinity capture media has the same or a similar concentration of denaturing agent as that found in the sample that is to be applied to the media. Equilibrating the column may substantially maintain denaturation of the target molecule(s) by maintaining the concentration of the denaturing agent after it is applied to the media. As an illustrative example, a chromatography column including anti-human serum albumin (HSA) resin may be washed with a solution of 2.25 M urea in PBS prior to the application of a serum sample that includes 2.25 M urea in PBS. Equilibrating the column may not be necessary if the fluid in the affinity capture media does not alter the concentration of the denaturing agent so as to compromise the denaturation of the target molecule(s).

In one embodiment, a sample is placed in contact with the at least one denaturing agent, e.g., a target molecule-containing sample fluid is combined with a denaturing agent-containing fluid, and a fluid containing the target molecule(s) and the denaturing agent(s) is subsequently placed in contact with the immobilized affinity molecule.

In another embodiment, a sample is placed in contact with an immobilized affinity molecule before the at least one denaturing agent is placed in contact with the sample and the immobilized affinity molecule. For example, a fluid containing at least one denaturing agent is added to a slurry comprising one more target molecules and one or more immobilized affinity molecules.

In yet another embodiment, at least one denaturing agent is placed in contact with an immobilized affinity molecule before a sample is placed in contact with the at least one denaturing agent and the immobilized affinity molecule. For example, a target molecule-containing sample fluid is added to a slurry comprising the at least one denaturing agent and one or more immobilized affinity molecules.

In still another embodiment, the target molecule-containing sample fluid, the at least one denaturing agent, and the one or more immobilized affinity molecules, are placed in contact with each other simultaneously, or essentially simultaneously.

Embodiments of the invention are suitable for use with any affinity capture protocol, including, but not limited to, enzyme-linked immunosorbent assays (ELISA), ELISPOT assays, immunoprecipitation assays, flow cytometry, agglutination reactions, immunodiffusion assays, immunoelectrophoresis assays, radioimmunoassays, Western blots, immunofluorescence assays, and immunoelectron microscopy, and are suitable for, but are not limited to, sample preparation, clinical diagnostic assays, and screening specimens, e.g., drugs in pharmaceutical research.

Embodiments of the invention can be adapted for use in a variety of techniques, including preparative methods employing fixed bed, fluidized bed, and batch chromatographies. Alternatively, embodiments can be practiced in the context of separation techniques, preferably high throughput separation techniques, that utilize devices such as spin columns or multiwell plate formats. If desired, such devices can be small devices where device volumes can be as small as measurable, e.g., a few nanoliters.

The invention can be used in any suitable setting, including, but not limited to, hospitals and laboratories.

Embodiments of the invention are suitable for use with a variety of samples and/or target molecules, e.g., to purify and/or concentrate one or more desired target molecules present in a fluid sample (for example, the affinity bound target molecule(s) can be subsequently eluted and recovered), and/or to provide a fluid sample depleted of one or more target molecules, e.g., for use in, but not limited to, proteomics applications. Embodiments of the invention include purifying and/or concentrating two or more different desired target molecules and/or providing a fluid sample depleted of two or more different target molecules.

Embodiments of the invention are applicable to affinity capturing a variety of target molecules, e.g., biological substances, which include proteins, peptides, viruses, nucleic acids, carbohydrates, and lipids. Preferably, the target molecule is a protein or peptide. More preferably, the protein is an immunoglobulin, albumin, hormone, clotting factor, cytokine, or enzyme. One more preferred protein is an immunoglobulin, e.g., a whole immunoglobulin, including monoclonal and polyclonal antibodies, as well as Fab, F(ab′)₂, Fc and Fv fragments thereof.

The biological substances typically derive from, or are contained in, sources including but not limited to liquid samples such as saliva, biological fluid, urine, lymphatic fluid, prostatic fluid, seminal fluid, milk, milk whey, organ extracts, plant extracts, cell extract, cell culture media, supernatants, fermentation broths, ascites fluid, lysates, transgenic plant and animal extracts, and buffers.

Embodiments of the invention can be suitable for treating process fluids such as fluids used in the biopharmaceutical industry, e.g., biotherapeutic fluids including desirable material such as proteinaceous material, for example, antibodies (e.g., monoclonal antibodies), recombinant proteins such as growth factors, or desired peptides, wherein the affinity captured desired material can be subsequently recovered. Alternatively, illustrative embodiments can be suitable for treating biological fluids to deplete the biological fluids of the target molecule(s). Illustratively, human serum albumin and/or human IgG can be depleted from serum and/or plasma samples. Since human serum albumin and human IgG can bind to other proteins while circulating in blood (and these protein-protein interactions can be sufficiently strong that they are maintained during blood collection, processing, and freeze-thaw cycles), depletion of either or both proteins from the biological fluid in accordance with embodiments of the invention reduces or minimizes the loss of other proteins that might otherwise bind to the albumin and/or IgG.

A biological fluid includes any treated or untreated fluid associated with living organisms, particularly blood, including whole blood, warm or cold blood, and stored or fresh blood; treated blood, such as blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC); blood products derived from blood or a blood component or derived from bone marrow; stem cells; red cells separated from plasma and resuspended in physiological fluid or a cryoprotective fluid; and platelets separated from plasma and resuspended in physiological fluid or a cryoprotective fluid. The biological fluid may have been treated to remove some of the leukocytes before being processed according to the invention. As used herein, blood product or biological fluid refers to the components described above, and to similar blood products or biological fluids obtained by other means and with similar properties.

In some embodiments wherein the target molecule containing fluid comprises human serum or plasma and is to be used more than once, the fluid can be separated into aliquots to avoid multiple freeze-thaw cycles, which can cause undesirable changes in proteins and/or increased precipitates.

If desired, the fluid (e.g., human serum or plasma) containing one or more target molecules is visually inspected for the presence of significant precipitate before being placed in contact with the denaturing agent. If significant precipitate is present, the target molecule containing fluid may be filtered and/or centrifuged before or after the liquid is diluted (e.g., wherein the target molecule containing fluid is mixed with a buffer to provide the liquid sample).

The liquid sample containing one or more target molecules is contacted with an immobilized single-chain affinity molecule in the presence of at least one denaturing agent for a period of time sufficient to allow at least one target molecule to bind to the immobilized affinity molecule. Typically, the contact period is between about 30 seconds to about 12 hours.

The target molecule depleted fluid is preferably separated from the immobilized affinity molecule (having target molecule(s) specifically bound thereto) by passing the depleted fluid from a device containing the immobilized affinity molecule. For example, the target molecule depleted fluid can be passed from a chromatography column (packed with beads or particles having the affinity molecule immobilized thereon), or from a multiple well plate (e.g., having porous media in the wells, wherein the affinity molecules are immobilized on the porous media). Alternatively, for example, the target molecule depleted fluid can be drawn off with a pipette, or passed from a spin column or multiple well plate (that can also comprise a spin device), wherein the column or plate retains beads or particles having the affinity molecule immobilized thereon, and allows the target molecule depleted fluid to pass from the column or plate.

If desired, the target molecule(s) can be eluted from the affinity molecule and recovered, e.g., to purify and/or concentrate the target molecule(s).

A variety of spin devices, preferably spin devices including separation media (e.g., including a membrane or frit with a pore size sufficient to prevent the passage of affinity molecule immobilized beads or particles therethrough), can be used in accordance with embodiments of the invention. Suitable commercially available spin devices include, for example, microfuge tubes, as well as spin columns such as centrifugal devices available from Pall Corporation (East Hills, N.Y.) as NANOSEP® Centrifugal Devices and MICROSEP™ Centrifugal Devices. Alternatively, suitable commercially available spin devices include, for example, multiple well centrifugal devices or multiple well plates, e.g., multiple well filter plates from Pall Corporation under the tradenames ACROWELL™ and ACROPREP™ and/or described in, for example, International Publication No. WO 2002/096563.

A variety of separation media are suitable for use in spin devices and/or multiple well plates in accordance with embodiments of the invention. Preferred media are membranes, in some embodiments, low protein binding membranes. The membranes can have any suitable porosity. In some embodiments, the membranes are microporous membranes. In some other illustrative embodiments, the membranes are ultrafiltration membranes, e.g., 10,000 molecular weight cut off (mwco) or greater, preferably, 30,000 mwco or greater, e.g., 50,000 mwco, or 100,000 mwco, or greater.

However, other embodiments of the invention do not require the use of spin devices. For example, as noted above, a chromatography column (e.g., packed with beads or particles with affinity molecules or antigen binding portions immobilized thereon) or a multiple well plate (e.g., wherein each well includes at least one porous medium (such as a microporous membrane) having affinity molecules or antigen binding portions immobilized thereon) can be utilized. Also as noted above, a multiple well plate can be utilized as a spin device or as a non-spin device.

In accordance with embodiments of the invention, a kit for affinity capture of a target molecule is provided comprising a support; a single-chain affinity molecule or antigen binding portion thereof, immobilized on the support; at least one buffer; and, a device suitable for containing the immobilized affinity molecule or antigen binding portion thereof.

In a preferred embodiment, the single-chain affinity molecule is a camelid antibody or an antigen binding portion thereof.

In some embodiments, the kit includes two or more single-chain affinity molecules or antigen binding portions thereof, wherein the molecules or antigen binding portions thereof have different binding specificities. For example, the kit can include a first single-chain affinity molecule or antigen binding portion thereof specific for one peptide or protein, for example, HSA (e.g., bound to one support or set of supports (e.g., beads)), and a second single-chain affinity molecule or antigen binding portion thereof specific for another peptide or protein, for example, IgG (e.g., bound to another support or set of supports (e.g., beads)).

Embodiments of the kit can further comprise one or more of the following: printed instructions for using the kit, one or more denaturing agents, one or more containers, e.g., a separate container for containing each of one or more buffers, affinity molecules bound to supports and/or a separate container for containing each of one or more denaturing agents.

In some embodiments of the kit, the support comprises a bead or particle, or a membrane, a fiber, or a biochip. Alternatively, or additionally, in some embodiments of the kit, the device suitable for containing the immobilized affinity molecule comprises a spin device and/or a multiple well plate.

The following examples further illustrate the invention but, of course, should not be construed as in any way limiting its scope.

In the following examples, the references to “low,” “medium,” and “high” concentrations of denaturing agent are merely used for ease of reference when referring to the various denaturing agents, e.g., the medium concentration referred to in an example merely means 3 concentrations are being referred to, and a medium concentration is less than another concentration used in the example, and is greater than yet another concentration used in the example.

### Example 1

This example demonstrates the depletion of HSA from a sample of human serum in the presence of different concentrations of denaturing agent.

In the first section of this Example, HSA is depleted from the serum in the presence of a medium concentration of denaturing agent.

A sample of human serum is prepared. In order to saturate the ligand to more easily notice even a small drop in depletion (corresponding to a small drop in affinity of the ligand for the target protein due to the presence of the denaturing agent), human serum is spiked with additional HSA (35 mg/mL) to obtain an HSA concentration close to the saturation level of the ligand, i.e., 50 mg/mL.

A batch of denaturing agent is prepared. High purity water is used to obtain a denaturing agent of 9 M urea+2% CHAPS in Tris buffer, pH 9.0.

A sample of the spiked human serum is treated with the denaturing agent. 50 μL of the spiked human serum described above is combined with 64 μL of 9 M urea+2% CHAPS in Tris buffer so that the final concentrations of urea and CHAPS are 5 M and 1.11%, respectively. The treated serum sample is incubated for 30 minutes at room temperature.

The sample with denaturing agent is further diluted. Phosphate buffered saline (PBS) buffer (136 μL) is added to the treated serum sample to obtain a volume of 250 μL having 2.25 M urea and 0.5% CHAPS prior to adding the treated serum sample to the slurry described below.

An anti-HSA slurry is prepared. Camelid antibodies specific for HSA are obtained under the trade name CAPTURESELECT® (BAC B.V., Naarden, The Netherlands). The antibodies are coupled to agarose beads by a glyoxyl reaction. The coupled beads are combined with storage buffer (PBS buffer comprising 0.02% azide) to provide a 50% slurry.

The slurry (400 μL) is transferred to a NANOSEP® MF Centrifugal Device (Pall Corporation, East Hills, N.Y.) including a GHP hydrophilic polypropylene membrane having a pore size of 0.45 μm, and centrifuged at 3,000 rpm for 2 minutes. The storage buffer is discarded.

The slurry is washed once with 400 μL of PBS buffer (pH 7.4) and vortexed. The vortexed slurry is centrifuged at 3,000 rpm for 2 minutes. The flow through wash solution is discarded.

A 250 μL sample of human serum denatured and diluted as described above is placed into the NANOSEP® device including the prepared slurry. The serum sample is mixed well by vortexing, and tumbled end over end for 15 minutes at room temperature. The mixed serum sample is centrifuged at 3,000 rpm for 1.5 minutes. The flow through is collected and provides the depleted sample.

The quantities of HSA in the initial sample, and depleted from the initial serum sample, are measured by enzyme-linked immunosorbent assay (ELISA). The ELISA is carried out using the Human Albumin ELISA Quantitation Kit (Bethyl Laboratories, Inc., Montgomery, Tex.), according to the manufacturer's instructions.

The amount of HSA depleted from the spiked sample is greater than 90%.

To deplete HSA from a human serum sample in the presence of a low concentration of denaturing agent, the procedures described above are generally followed, except that a human serum sample comprising 1.5 M urea and 0.33% CHAPS (a 50 μL spiked sample is combined with 42 μL of 9 M urea+2% CHAPS, and PBS is added to obtain a volume of 250 μL) is applied to the prepared slurry. The amount of HSA depleted from the serum in the presence of a low concentration of denaturing agent is measured by ELISA as described above is greater than 90%.

To deplete HSA from a human serum sample in the presence of a high concentration of denaturing agent, the procedures described above are generally followed, except that a human serum sample comprising 3 M urea and 0.67% CHAPS (a 50 μL spiked sample is combined with 83 μL 9 M urea+2% CHAPS, and PBS is added to obtain a volume of 250 μL) is applied to the prepared slurry. The amount of HSA depleted from the serum in the presence of a low concentration of denaturing agent is measured by ELISA as described above and is greater than 90%.

To deplete HSA from a human plasma sample in the presence of each of low, medium, and high concentrations of denaturing agent, the procedures described above are followed substituting human plasma for human serum. The amount of HSA depleted from the plasma is measured by ELISA as described above and is greater than 90% in the presence of each of low, medium and high concentrations of denaturing agent.

### Example 2

In this Example (a control) HSA is depleted from human serum in the absence of denaturing agent. When compared to results obtained from the depletion of HSA in the presence of denaturing agent in Example 1, this Example demonstrates that the depletion of HSA from human serum in the presence of denaturing agent does not significantly reduce the affinity of the camelid antibodies for HSA.

The procedures described in Example 1 are followed in which 200 μL of PBS is added to 50 μL of serum sample and no denaturing agent is added to the serum. The 250 μL of serum and PBS is added to the NANOSEP® device containing the prepared slurry as described in Example 1.

The amount of HSA depleted from the serum is measured by ELISA as described in Example 1 and is found to be greater than 90%.

To demonstrate that the depletion of HSA from human plasma in the presence of denaturing agent does not significantly reduce the affinity of the camelid antibodies for HSA, the procedures described in this Example are followed substituting human plasma for human serum. The amount of HSA depleted from the plasma is measured by ELISA as described in Example 1 and is greater than 90%.

### Example 3

This example demonstrates the depletion of IgG from human serum in the presence of different concentrations of a denaturing agent.

In the first section of this Example, IgG is depleted from the serum in the presence of a medium concentration of denaturing agent.

A sample of human serum is prepared. Human serum is spiked with additional IgG (7 mg/mL) to obtain an IgG concentration of 14.7 mg/mL.

An anti-IgG slurry is prepared. Camelid antibodies specific for IgG (CAPTURESELECT®) are coupled to agarose beads and combined with buffer as described in Example 1 to provide an anti-IgG slurry.

The spiked human serum is denatured and depleted as described in Example 1.

The quantity of IgG initially present, and depleted from the serum sample, is measured by ELISA. The ELISA is carried out using the Human IgG ELISA Quantitation Kit (Bethyl Laboratories, Inc.), according to the manufacturer's instructions. The amount of IgG depleted from the spiked serum is greater than 95%.

To deplete IgG from a human serum sample in the presence of a low concentration of denaturing agent, the procedures in described above are generally followed, except that a human serum sample comprising 1.5 M urea and 0.33% CHAPS is applied to the prepared slurry. The amount of IgG depleted from the serum is measured by ELISA as described above and is greater than 95%.

To deplete IgG from a human serum sample in the presence of a high concentration of denaturing agent, the procedures described above are generally followed, except that a human serum sample comprising 3 M urea and 0.67% CHAPS is applied to the prepared slurry. The amount of IgG depleted from the serum is measured by ELISA as described above and is greater than 95%.

To deplete IgG from a human plasma sample in the presence of each of low, medium, and high concentrations of denaturing agent, the procedures described in this Example are followed substituting human plasma for human serum. The human plasma is spiked with 7 mg/mL additional IgG to reach an IgG concentration of 16.1 mg/mL. The amount of IgG depleted from the plasma is measured by ELISA as described above and is greater than 95%.

### Example 4

In this Example (a control) IgG is depleted from human serum in the absence of denaturing agent. When compared to results obtained from the depletion of IgG in the presence of denaturing agent in Example 3, this Example demonstrates that the depletion of IgG from human serum in the presence of denaturing agent does not significantly reduce the affinity of the camelid antibodies for IgG.

The procedures described in Example 3 are followed in which 200 μL of PBS is added to 50 μL of serum sample and no denaturing agent is added to the serum. The 250 μL of serum and PBS is added to the NANOSEP® device containing the anti-IgG slurry as described in Example 3.

The amount of IgG depleted from the serum is measured by ELISA as described in Example 3 and is greater than 95%.

To demonstrate that the depletion of IgG from human plasma in the presence of denaturing agent does not significantly reduce the affinity of the camelid antibodies for IgG, the procedures described in this Example are followed substituting human plasma for human serum. The amount of IgG depleted from the plasma is measured by ELISA as described in Example 3 and is greater than 95%.

### Example 5

This example demonstrates the depletion of both IgG and HSA proteins from the same sample of human serum in the presence of a denaturing agent.

A sample of human serum is prepared. Human serum is spiked with additional HSA (35 mg/mL) and additional IgG (7 mg/mL) to obtain an HSA concentration of 50 mg/mL and an IgG concentration of 14.73 mg/mL.

The spiked serum sample is treated with medium concentration denaturing agent and diluted with PBS buffer as described in Example 1 to obtain a volume of 250 μL having 2.25 M urea and 0.5% CHAPS.

An anti-HSA slurry is prepared as described in Example 1, and an anti-IgG slurry is prepared as described in Example 3.

The anti-HSA slurry (400 μL) is transferred to a NANOSEP® device and centrifuged as described in Example 1. The storage buffer is discarded. The anti-IgG slurry (400 μL) is transferred to the NANOSEP® device containing the anti-HSA slurry and centrifuged as described in Example 1. The storage buffer is again discarded.

The slurry is washed with PBS buffer, vortexed, and centrifuged as described in Example 1. The flow through wash solution is discarded.

A 250 μL sample of human serum that is denatured and diluted as described above is placed into the NANOSEP® device including the prepared anti-HSA/anti-IgG slurry. The serum sample is mixed, tumbled end over end, and centrifuged as described in Example 1. The flow through is collected and provides the depleted sample.

The quantities of HSA and IgG initially present, and depleted from the initial serum sample, are measured by ELISA as described in Examples 1 and 3, respectively. The amount of HSA depleted from the spiked serum sample as described above is greater than 90%. The amount of IgG depleted from the spiked serum sample as described above is greater than 95%.

To deplete HSA and IgG from a human serum sample in the presence of a low concentration of denaturing agent, the procedures in this Example are generally followed, except that a human serum sample comprising 1.5 M urea and 0.33% CHAPS is applied to the prepared slurry. The amount of HSA depleted from the serum is measured by ELISA as described above and is greater than 90%. The amount of IgG depleted from the serum is measured by ELISA as described above and is greater than 95%.

To deplete HSA and IgG from a human serum sample in the presence of a high concentration of denaturing agent, the procedures described above are generally followed, except that a human serum sample comprising 3 M urea and 0.67% CHAPS is applied to the prepared slurry. The amount of HSA depleted from the serum is measured by ELISA as described above and is greater than 90%. The amount of IgG depleted from the serum is measured by ELISA as described above and is greater than 95%.

To deplete both HSA and IgG, combined, from a human plasma sample in the presence of each of low, medium, and high concentrations of denaturing agent, the procedures in this Example are followed substituting human plasma for human serum. The amount of HSA depleted from the plasma is measured by ELISA as described above and is greater than 90% in the presence of each of low, medium and high concentrations of denaturing agent. The amount of IgG depleted from the plasma is measured by ELISA as described above and is greater than 95% in the presence of each of low, medium and high concentrations of denaturing agent.

### Example 6

In this Example (a control) HSA and IgG are both depleted from human serum in the absence of denaturing agent. When compared to results obtained from the depletion of both HSA and IgG in the presence of denaturing agent in Example 5, this Example demonstrates that the depletion of both HSA and IgG from human serum in the presence of denaturing agent does not significantly reduce the affinity of the camelid antibodies for HSA or IgG.

The procedures described in Example 5 are followed in which 200 μL of PBS is added to 50 μL of serum sample and no denaturing agent is added to the serum. The 250 μL of serum and PBS is added to the NANOSEP® device containing the anti-HSA/anti-IgG slurry as described in Example 5.

The amount of HSA depleted from the serum is measured by ELISA as described above and is found to be greater than 90%. The amount of IgG depleted from the serum is measured by ELISA as described above and is greater than 95%.

To demonstrate that the that the depletion of HSA and IgG, combined, from human plasma in the presence of denaturing agent does not significantly reduce the affinity of the camelid antibodies for HSA, the procedures described in this Example are followed substituting human plasma for human serum. The amount of HSA depleted from the plasma is measured by ELISA as described above and is found to be greater than 90%. The amount of IgG depleted from the plasma is measured by ELISA as described above and is greater than 95%.

In carrying out the Example 7, the amount of protein bound is determined as follows: Excess pure HSA is loaded onto the anti-HSA slurry (camelid antibodies coupled to agarose beads). The amount of HSA that does not bind is determined and used to calculate the amount bound (amount loaded minus amount that flows through the device+anti-HSA slurry). Comparison of the amount of HSA bound in the absence or presence of various denaturing agents demonstrates the effect (positive, neutral, or negative) of each condition.

Additionally, the same protocol is carried out using BSA, to measure non-specific binding.

Example 7 is carried out as follows, at room temperature.

In preparation for determining specific binding, the protein solution (40 mg/ml HSA) is prepared by dissolving 40 mg HSA in 1.0 ml of 1×PBS in a 1.5 ml micro-centrifuge tube. The solution is rotated for 30 minutes.

In preparation for determining non-specific binding, 40 mg/ml BSA is prepared in a similar manner.

Five different buffered denaturing agents are prepared as described following the “Procedure” section below. Binding is carried out as described in the following procedure.

### Procedure

(a) 200 μL of the anti-HSA resin (50% slurry, as described in Example 1) is pipetted into a 1.5 mL micro-centrifuge tube.

(b) The slurry is centrifuged at 900 g for 1.5 min to remove the storage buffer (PBS buffer comprising 0.02% azide), resulting in 100 μL of packed resin.

(c) The slurry is resuspended in 400 μL of PBS (wash buffer) and the tube is rotated for 5 minutes to wash the beads.

(d) The tube is centrifuged at 900 g for 1.5 min to remove the wash buffer.

(e) Steps (a)-(d) are repeated for 2 washes.

(f) The beads are resuspended in 400 μL of the buffered denaturing agent to be tested and the tube is rotated for 5 minutes to equilibrate the beads.

(g) The tube is centrifuged at 900 g for 1.5 min to remove the buffered denaturing agent.

(h) 50 μL of the protein solution and 200 μL of the buffered denaturing agent are incubated for 15 minutes with rotation. Additionally, in order to determine native binding, in a separate tube, 50 μL of the protein solution and either 200 μL of the buffer without denaturing agent (PBS without: CTAB, guanidine HCl, urea/CHAPS, or acetonitrile) are incubated for 15 minutes with rotation.

(i) The diluted sample is added to the anti-HSA resin, and the sample is incubated with the beads by rotating the tube for 1 hour.

(j) The sample is centrifuged at 900 g for 1.5 min and the flow through is collected.

(k) The beads are washed with 400 μL of PBS (wash buffer) with rotation for 10 minutes, and centrifuged at 900 g for 1.5 min to remove and collect the wash buffer.

The flow through and wash buffers for each test are pooled and contain the unbound HSA. Similarly, the flow through and wash buffers for each test are pooled and contain the unbound BSA.

The amount of unbound HSA is determined by measuring the protein concentration of the pooled samples using the BCA protein assay (Pierce BCA™ Protein assay kit #23227) according to the manufacturer's instructions.

The total amount of unbound HSA=[HSA]×volume pooled sample.

Nonspecific binding of BSA=[BSA]×volume pooled sample.

HSA capacity=[(amount HSA loaded)−(amount unbound HSA)]−[(amount of BSA loaded)−(amount of unbound BSA)]. Nonspecific binding is determined for each denaturing agent and native binding condition tested.

### Example 7

This example demonstrates the binding of purified HSA in the presence of five different buffered denaturing agents, i.e., CTAB (1.6%), guanidine HCl (0.4 M and 0.8 M), acetonitrile (ACN) (8% and 16%), 8.1 M urea+1.6% CHAPS, and 80 mM (0.08 M) sodium acetate (NaOAc) pH 4.5, and, for comparison, the binding of purified HSA in the presence of a non-denaturing buffer (PBS).

2% CTAB is prepared by dissolving 2 g of CTAB in 100 mL PBS.

0.5 M guanidine HCl is prepared by dissolving 4.78 g guanidine HCl in PBS (pH 7.2) for a final volume of 100 mL.

1.0 M guanidine HCl is prepared by dissolving 9.56 g guanidine HCl in PBS (pH 7.2) for a final volume of 100 mL.

10% acetonitrile is prepared by adding PBS (pH 7.2) to 10 mL of acetonitrile to a final volume of 100 mL.

20% acetonitrile is prepared by adding PBS (pH 7.2) to 20 mL of acetonitrile to a final volume of 100 mL.

9 M urea/2% CHAPS is prepared by adding 5.48 g of urea and 2 g of CHAPS to PBS (pH 7.2) for a final volume of 100 mL.

100 mM (0.1 M) sodium acetate is prepared by adding 13.61 g of sodium acetate to 800 mL dH₂O, the pH is adjusted to 4.5 by NaOH, and dH₂O is added to bring it to 1 L.

The buffered denaturing agents are mixed with albumin in solution, resulting in a 20% decrease in final denaturing agent concentration (i.e., 5-fold dilution of sample) during the binding step.

Binding is carried out as described in the “Procedure” section above.

As shown below, the HSA binding capacity under non-denaturing conditions (i.e., binding in PBS) is 8.0 mg/ml. The HSA binding capacity in the presence of 16% acetonitrile (7.5 mg/ml), 0.08 M NaOAC pH 4.5 (8.9 mg/ml), and 8.1 M urea/1.6% CHAPS (8.4 mg/ml) is similar to native binding capacity, indicating no significant effect of the denaturing agent.

As shown below, the HSA binding capacity in the presence of 8% acetonitrile (6.3 mg/ml), 0.4 M guanidine HCl (5.4 mg/ml), 0.8 guanidine HCl (4.8 mg/ml) and 1.6% CTAB (5.7 mg/ml) is somewhat lower than native binding capacity, but still shows significant binding, and thus, there is limited disruption of the target protein and camelid antibody interaction.

| | Initial | Final | Capacity | % Native |
|---|---|---|---|---|
| Buffer | Concentration | Concentration | (mg/ml) | Capacity |
| PBS | 1x | 1x | 8.0 | |
| ACN-PBS | 10% | 8% | 6.3 | 78.1 |
| ACN-PBS | 20% | 16% | 7.5 | 93.7 |
| guanidine HCl- | 0.5 M | 0.4 M | 5.4 | 67.1 |
| PBS | | | | |
| guanidine HCl- | 1.0 M | 0.8 M | 4.8 | 59.9 |
| PBS | | | | |
| CTAB-PBS | 2% | 1.6% | 5.7 | 70.5 |
| NaOAc pH 4.5 | 100 mM | 80 mM | 8.9 | 111.4 |
| Urea/CHAPS- | 9 M/2% | 8.1 M/1.6% | 8.4 | 104.6 |
| PBS | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for affinity capture of a target molecule comprising:
obtaining a sample comprising a target molecule; and,
in the presence of a denaturing agent;
affinity capturing the target molecule with a single-chain affinity molecule or antigen binding portion thereof that specifically binds the target molecule, wherein the affinity molecule or antigen binding portion thereof is immobilized on a support.

2. The method of claim 1, wherein the denaturing agent further comprises a detergent.

3. The method of claim 1, wherein the support comprises a bead.

4. The method of claim 3, wherein the bead is disposed in a chromatography column.

5. The method of claim 3, wherein the bead is disposed in a spin device.

6. The method of claim 3, wherein the bead is disposed in a multiple well plate.

7. The method of claim 1, wherein the support comprises a membrane.

8. The method of claim 7, wherein the membrane is disposed in a multiple well device.

9. The method of claim 7, wherein the membrane is disposed in a spin device.

10. The method of claim 1, comprising combining the sample with the denaturing agent before placing the sample and the denaturing agent in contact with the affinity molecule.

11. The method of claim 1, comprising placing the sample in contact with the affinity molecule before placing the denaturing agent in contact with the sample and the affinity molecule.

12. The method of claim 1, comprising placing the denaturing agent in contact with the affinity molecule before placing the sample in contact the affinity molecule and the denaturing agent.

13. The method of claim 1, further comprising eluting the target molecule from the affinity molecule and recovering the eluted target molecule.

14. The method of claim 1, further comprising obtaining a target molecule-depleted fluid.

15. The method of claim 1, wherein the affinity molecule is a camelid antibody or a fragment thereof.

16. The method of claim 1, wherein the target molecule is a protein.

17. The method of claim 1, wherein the target molecule is a peptide.

18. The method of claim 1, wherein the denaturing agent is selected from the group consisting of urea, CHAPS, CTAB, guanidine HCL, acetonitrile, and acetate.

19. A kit comprising:
a support;
a single-chain affinity molecule or antigen binding portion thereof, immobilized on the support;
at least one buffer; and,
a device suitable for containing the immobilized affinity molecule or antigen binding portion thereof.

20. The kit of claim 19, comprising at least first and second single-chain affinity molecules or antigen binding portions thereof, the first single-chain affinity molecule having a different binding specificity than the second single-chain affinity molecule.

21. The kit of claim 20, wherein the first and second single-chain affinity molecules or antigen binding portions thereof are immobilized on separate supports.

22. The kit of claim 19, further comprising printed instructions for using the kit.

23. The kit of claim 19, comprising a biochip comprising at least one single-chain affinity molecule or antigen binding portion thereof, immobilized on the support.

24. The kit of claim 19, further comprising a container containing the single-chain affinity molecule or antigen binding portion thereof immobilized on the support.

25. The kit of claim 19, comprising a first container containing the first single-chain affinity molecule or antigen binding portion thereof immobilized on a support, and a second container containing the second single-chain affinity molecule or antigen binding portion thereof immobilized on a support.

26. The kit of claim 19, further comprising a denaturing agent.

27. The kit of claim 19, further comprising a spin device.

28. The method of claim 15, wherein the target molecule is albumin.

29. The method of claim 15, wherein the target molecule is IgG.

30. The method of claim 1, wherein the sample includes two or more different target molecules, and the method comprises affinity capturing at least two different target molecules.
